(19) United States
(12) Patent Application Publication
(10) Pub. No.: US 2022/0378720 A1
(43) Pub. Date: Dec. 1, 2022

(54) Composition for Improving or Maintaining Quality of Life

(71) Applicant: House Wellness Foods Corporation, Hyogo (JP)

(72) Inventors: Ryusei Uchio, Itami-shi, Hyogo (JP); Koutarou Muroyama, Itami-shi, Hyogo (JP); Kengo Kawasaki, Itami-shi, Hyogo (JP); Shinji Murosaki, Itami-shi, Hyogo (JP)

(73) Assignee: House Wellness Foods Corporation, Hyogo (JP)

(21) Appl. No.: 17/623,298

(22) PCT Filed: Jun. 28, 2019
(86) PCT No.: PCT/JP2019/025840

§ 371 Date: Dec. 28, 2021

(51) Int. Cl. A61K 31/12; A61K 36/9066

(52) U.S. Cl. CPC A61K 31/12; A61K 36/9066; A61K 2236/17

(57) ABSTRACT

The present invention provides a composition for improving or maintaining quality of life (QOL), comprising, as an active ingredient, a component derived from turmeric. The present invention relates to a composition for improving or maintaining QOL, comprising, as an active ingredient, at least one of turmeronol A, turmeronol B, and bisacurone. The composition of the present invention for improving or maintaining QOL may comprise 20 μg or more of turmeronol A and turmeronol B in total per daily intake, may comprise 17 μg or more of turmeronol A per daily intake, may comprise 5 μg or more of turmeronol B per daily intake, and may comprise 80 μg or more of bisacurone per daily intake.

### TECHNICAL FIELD

The present invention relates to a composition for improving or maintaining quality of life (QOL).

### BACKGROUND ART

Quality of life (QOL) is defined by World Health Organization as “a state of complete physical, mental, and social well-being and not merely the absence of disease” (Non Patent Literature 1). QOL is known to vary in a manner depending on the physical, mental, social, and economic condition of a human, and worsens in various bad situations involving bad health condition, such as aging, disease, and need of care (Non Patent Literature 2). Therefore, improvement of QOL is expected to lead to promotion and maintenance of health for humans.

Patent Literature 1 discloses that a specific strain of the genus Lactobacillus has an effect of improving or maintaining QOL. Patent Literature 1 discloses that scores of QOL based on SF-36v2 (Registered Trademark) were improved for test subjects who had ingested the specific strain of the genus Lactobacillus.

Patent Literature 2 discloses that a hepatitis C patient who had ingested an herbal composition containing extracts of flowers of yellow cordia, leaves of soursop, and roots of turmeric achieved improved QOL. In Examples of Patent Literature 2, ethanol-water extracts of roots of turmeric are used as extracts of roots of turmeric.

### CITATION LIST

### Patent Literature

Patent Literature 1: JP Patent Publication (Kokai) No. 2017-19841 A
Patent Literature 2: JP Patent Publication (Kohyo) No. 2010-540572 A

### Non Patent Literature

Non Patent Literature 1: Yuriko DOI, Introduction-Conceptual Issues on Quality of Life (QOL) and Importance of QOL Research, J. Natl. Inst. Public Health, 53(3), 2004
Non Patent Literature 2: Shunichi FUKUHARA, Yoshimi SUZUKAMO (2015) “SF-36v2 manual, Japanese version”, iHOPE International

### SUMMARY OF INVENTION

### Technical Problem

An object of the present invention is to provide a composition effective for improving or maintaining QOL.

### Solution to Problem

The present inventors have found that QOL was significantly improved or maintained in humans who had ingested tablets each comprising a turmeric extract comprising turmeronol A, turmeronol B, and bisacurone every day for 12 weeks. This has led to the completion of the present invention described below.

(1) A composition for improving or maintaining quality of life (QOL), comprising, as an active ingredient, at least one of turmeronol A, turmeronol B, and bisacurone.
(2) The composition for improving or maintaining quality of life (QOL) according to (1), wherein the composition comprises a turmeric extract comprising the at least one of turmeronol A, turmeronol B, and bisacurone and extracted with at least one extraction solvent selected from the group consisting of water and a hydrophilic organic solvent.
(3) A composition for improving or maintaining quality of life (QOL), comprising, as an active ingredient, a turmeric extract comprising at least one of turmeronol A, turmeronol B, and bisacurone and extracted with at least one extraction solvent selected from the group consisting of water and a hydrophilic organic solvent.
(4) The composition for improving or maintaining quality of life (QOL) according to any one of (1) to (3), the composition does not comprise one or more plants belonging to a genus selected from the genus Cordia and the genus Annona.
(5) The composition for improving or maintaining quality of life (QOL) according to any one of (1) to (4), for administering to a subject not affected by hepatitis.
(6) The composition for improving or maintaining quality of life (QOL) according to (5), wherein the subject is a healthy human.
(7) The composition for improving or maintaining quality of life (QOL) according to any one of (1) to (6), wherein the composition comprises 20 μg or more of turmeronol A and turmeronol B in total per daily intake.
(8) The composition for improving or maintaining quality of life (QOL) according to any one of (1) to (7), wherein the composition comprises 17 μg or more of turmeronol A per daily intake.
(9) The composition for improving or maintaining quality of life (QOL) according to any one of (1) to (8), wherein the composition comprises 5 μg or more of turmeronol B per daily intake.
(10) The composition for improving or maintaining quality of life (QOL) according to any one of (1) to (9), wherein the composition comprises 80 μg or more of bisacurone per daily intake.
(11) The composition for improving or maintaining quality of life (QOL) according to any one of (1) to (10), wherein a content of curcumin is less than 30 mg per daily intake.
(12) Use of at least one of turmeronol A, turmeronol B, and bisacurone, or a turmeric extract comprising at least one of turmeronol A, turmeronol B, and bisacurone and extracted with at least one extraction solvent selected from the group consisting of water and a hydrophilic organic solvent, for production of a composition for improving or maintaining quality of life (QOL).
(13) Use of at least one of turmeronol A, turmeronol B, and bisacurone, or a turmeric extract comprising at least one of turmeronol A, turmeronol B, and bisacurone and extracted with at least one extraction solvent selected from the group consisting of water and a hydrophilic organic solvent, for production of a medicament for improving or maintaining quality of life (QOL).

The composition or medicament for improving or maintaining QOL preferably does not comprise one or more plants belonging to a genus selected from the genus Cordia and the genus Annona. The composition or medicament for improving or maintaining QOL is preferably a composition or medicament for administering to a subject not affected by hepatitis, in particular, to a healthy human. The composition or medicament for improving or maintaining QOL preferably comprises 20 μg or more of turmeronol A and turmeronol B in total per daily intake. The composition or medicament for improving or maintaining QOL preferably comprises 17 μg or more of turmeronol A per daily intake. The composition or medicament for improving or maintaining QOL preferably comprises 5 μg or more of turmeronol B per daily intake. The composition or medicament for improving or maintaining QOL preferably comprises 80 μg or more of bisacurone per daily intake. The content of curcumin in the composition or medicament for improving or maintaining QOL is preferably less than 30 mg per daily intake.

(14) A method for improving or maintaining quality of life (QOL), comprising:

administering at least one of turmeronol A, turmeronol B, and bisacurone, or a turmeric extract comprising at least one of turmeronol A, turmeronol B, and bisacurone and extracted with at least one extraction solvent selected from the group consisting of water and a hydrophilic organic solvent, to a subject in need of improving or maintaining quality of life (QOL); and

improving or maintaining quality of life (QOL) in the subject.

(15) At least one of turmeronol A, turmeronol B, and bisacurone, or a turmeric extract comprising at least one of turmeronol A, turmeronol B, and bisacurone and extracted with at least one extraction solvent selected from the group consisting of water and a hydrophilic organic solvent, for improving or maintaining quality of life (QOL) in a subject in need of improving or maintaining quality of life (QOL).

The active compound or the turmeric extract in (15) is preferably administered to the subject without being used in combination with one or more plants belonging to a genus selected from the genus Cordia and the genus Annona. The active compound or the turmeric extract in (15) is administered, preferably, to a subject not affected by hepatitis, in particular, to a healthy human. The active compound or the turmeric extract in (15) is for improving or maintaining QOL by administering, preferably, 20 μg or more of turmeronol A and turmeronol B in total to the subject per day. The active compound or the turmeric extract in (15) is for improving or maintaining QOL by administering, preferably, 17 μg or more of turmeronol A to the subject per day. The active compound or the turmeric extract in (15) is for improving or maintaining QOL by administering, preferably, 5 μg or more of turmeronol B to the subject per day. The active compound or the turmeric extract in (15) is for improving or maintaining QOL by administering, preferably, 80 μg or more of bisacurone to the subject per day. The active compound or the turmeric extract in (15) is for improving or maintaining QOL by administering to the subject, preferably, so that the daily dose of curcumin is less than 30 mg.

### Advantageous Effects of Invention

The present invention provides a composition for improving or maintaining QOL.

### BRIEF DESCRIPTION OF DRAWINGS

FIG. 1 shows amounts of change (ΔGH) in general health (GH) scores from before intake of a test food (a turmeric extract group or a placebo group) to 4 weeks, 8 weeks, and 12 weeks after the intake.

FIG. 2 shows amounts of change (ΔVT) in vitality (VT) scores from before intake of a test food (a turmeric extract group or a placebo group) to 4 weeks, 8 weeks, and 12 weeks after the intake.

FIG. 3 shows amounts of change (ΔMH) in mental health (MH) scores from before intake of a test food (a turmeric extract group or a placebo group) to 4 weeks, 8 weeks, and 12 weeks after the intake.

FIG. 4 shows amounts of change (ΔMCS) in mental component summary (MCS) scores from before intake of a test food (a turmeric extract group or a placebo group) to 4 weeks, 8 weeks, and 12 weeks after the intake.

### DESCRIPTION OF EMBODIMENTS

### <Turmeric>

In the present invention, the term “turmeric” refers to a plant belonging to the genus Curcuma in the family Zingiberaceae, and specific examples thereof include Curcuma longa (autumn turmeric), Curcuma aromatica, Curcuma zedoaria, Curcuma phaeocaulis, Curcuma kwangsiensis, Curcuma wenyujin, and/or Curcuma xanthorrhiza. Among these, Curcuma longa is particularly preferable.

In the present invention, use of a portion of turmeric including the rhizome is preferable.

The form of turmeric for use in the present invention is not limited, and may be a crushed product, squeezed juice, or extract of turmeric, or a processed product thereof. Particularly preferable is a turmeric extract. In the following, preferred embodiments of the turmeric extract will be described.

### <Turmeric Extract>

In the present invention, the term “turmeric extract” refers to an extract of a plant material derived from a plant of the genus Curcuma in the family Zingiberaceae, the extract being extracted with an extraction solvent. The turmeric extract is not limited to a solvent extract obtained via extraction with an extraction solvent, and the scope of the turmeric extract includes a resultant of additional fractionation or purification through column chromatography or the like for such a solvent extract. The turmeric extract for use in the present invention can be in the form of an extract solution after the completion of an extraction procedure (including a fractionation or purification procedure if fractionation or purification is performed), a concentrate obtained by partially removing the solvent from the extract solution, or a dry matter obtained by removing the solvent from the extract solution. The solvent can be removed from the extract by volatilizing the solvent via, for example, heating and/or decompressing. The methods for such heating and decompressing are not limited, and, for example, known methods can be employed.

Examples of the above plant material include the aforementioned turmeric rhizome. For example, a rhizome of Curcuma longa is particularly preferable. The rhizome for use may be one collected from soil. An adequate part of the rhizome in its original form, in pieces obtained by cutting into adequate dimensions or shape, or in the form of a grounded product may be used. The plant material may have been dried, as appropriate.

As an extraction solvent, at least one selected from the group consisting of water and a hydrophilic organic solvent can be used. The at least one extraction solvent selected from the group consisting of water and a hydrophilic organic solvent may be any of water, a hydrophilic organic solvent, and a mixed solvent of water and a hydrophilic organic solvent. The hydrophilic organic solvent may be a mixed solvent of a plurality of hydrophilic organic solvents. The scope of “water” includes hot water. As such hot water, for example, hot water with a temperature of 95° C. or higher can be used. Examples of the hydrophilic organic solvent include at least one alcohol (which may be a mixed solvent of a plurality of alcohols), and the alcohol is preferably, but not limited to, ethanol. In the case that a mixed solvent of alcohol and water is used as the extraction solvent, the mixing ratio is preferably in the range of 10:90 to 90:10, and more preferably in the range of 20:80 to 50:50, for example, at a weight ratio, though the mixing ratio is not limited thereto.

Further, supercritical carbon dioxide can be used as the extraction solvent.

The extraction method to obtain the turmeric extract from the plant material is not limited.

In the present invention, for the turmeric extract, use of a turmeric extract comprising at least one of turmeronol A, turmeronol B, and bisacurone and extracted with the above extraction solvent is preferable, use of a turmeric extract comprising at least turmeronol A and turmeronol B and extracted with the above extraction solvent is more preferable, and use of a turmeric extract comprising turmeronol A, turmeronol B, and bisacurone and extracted with the above extraction solvent is further preferable.

Turmeronol A and turmeronol B are each a compound having a planar structure shown below.

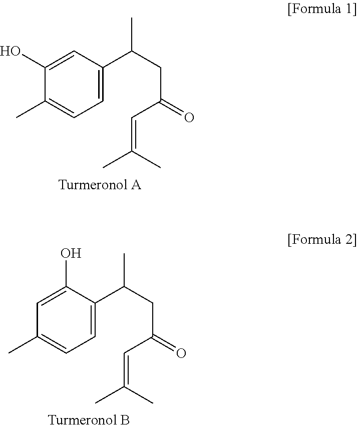 [SMILES: CC(C)=CC(=O)CC(C)c1ccc(C)c(O)c1 ; CC(C)=CC(=O)CC(C)c1ccc(C)cc1O]

In turmeronol A and turmeronol B in a naturally-occurring substance separated from turmeric, the configuration of the carbon at position 6 of the partial structure 2-methyl-2-hepten-4-one is S configuration. In the present invention, however, it is sufficient for each of turmeronol A and turmeronol B to have the corresponding planar structure shown above, and the configuration may be S configuration, R configuration, or a mixture of S and R configurations.

In the present invention, bisacurone is a compound having a planar structure shown below. Bisacurone has an asymmetric carbon at the position indicated with the symbol * in the formula of the planar structure, and there may be multiple optical isomers of bisacurone. In the present invention, bisacurone may be any optical isomer thereof, or a mixture of two or more optical isomers thereof.

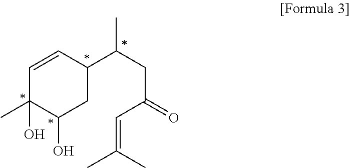 [SMILES: CC(C)=CC(=O)CC(C)C1C=CC(C)(O)C(O)C1]

### <Turmeronol A, Turmeronol B, and Bisacurone>

At least one of turmeronol A, turmeronol B, and bisacurone (hereinafter, also referred to as the “active compound”) for use in the present invention may be originated from a plant, or artificially synthesized. For example, optically active (+)-turmeronol A can be synthesized with a method described in Biosci Biotechnol Biochem. 1993; 57(7): 1137-40.

In the present invention, it is sufficient for each of turmeronol A, turmeronol B, and bisacurone to have the corresponding planar structure shown above. Each of turmeronol A, turmeronol B, and bisacurone may be any optical isomer thereof, or a mixture of a plurality of optical isomers thereof.

The active compound for use in the present invention is more preferably originated from a plant material, and further preferably originated from a plant of the genus Curcuma in the family Zingiberaceae. Specific examples of plants of the genus Curcuma in the family Zingiberaceae and parts thereof are as described above. The active compound can be obtained from a part, such as a rhizome, of a plant of the genus Curcuma in the family Zingiberaceae.

The active compound can be extracted from a plant material containing it. The extraction method is as described above. The active compound may be in the form of a plant extract, in particular, a turmeric extract extracted with at least one extraction solvent selected from the group consisting of water and a hydrophilic organic solvent.

Alternatively, a fraction of the active compound highly purified from a plant extract containing the active compound may be used in the present invention, and such a fraction may be blended in the composition of the present invention. For example, the active compound can be highly purified in an ethyl acetate fraction by subjecting a plant extract containing the active compound to liquid-liquid distribution using ethyl acetate/water. Alternatively, a plant extract containing the active compound or a fraction thereof may be subjected to a purification process via chromatography to obtain the active compound as a highly purified product. Examples of chromatography techniques that can be employed include reversed-phase column chromatography and normal-phase thin-layer chromatography.

A plant extract containing the active compound or a fraction thereof may be subjected to further processing, such as dehydration, pulverization, granulation, and solubilization, in accordance with a conventional technique.

The active compound preferably contains at least turmeronol A and turmeronol B, and more preferably contains turmeronol A, turmeronol B, and bisacurone.

### <Composition for Improving or Maintaining QOL>

The composition of the present invention for improving or maintaining QOL has an effect of improving or maintaining QOL, specifically, an effect of improving physical health and/or mental health or inhibiting the worsening thereof. The composition of the present invention for improving or maintaining QOL can improve or maintain QOL with respect to the physical aspect and/or mental aspect, and can be used as an agent for improving or maintaining physical and/or mental health. In particular, the composition of the present invention for improving or maintaining QOL has an effect of enhancing sense of well-being in general health, vitality, and sense of well-being in mental health. Hence, the composition of the present invention for improving or maintaining QOL can be used as an agent for improving or maintaining vitality, an agent for fatigue recovery, an agent for fatigue relief, or the like.

In evaluation of QOL, MOS 36-Item Short-Form Health Survey (SF-36 (Registered Trademark)), a validated survey method for evaluating health-related QOL in humans, is widely used to assess health condition for a wide variety of individuals including healthy and sick individuals (Non Patent Literature 2). SF-36v2 (Registered Trademark) is an improved version of SF-36 (Registered Trademark). In Examples, QOL was evaluated on the basis of SF-36v2 (Registered Trademark). The composition of the present invention for improving or maintaining QOL has an effect of increasing scores in one or more, preferably two or more, more preferably three or more, most preferably four items selected from general health (GH), vitality (VT), mental health (MH), and mental component summary (MCS) according to SF-36v2 (Registered Trademark), or inhibiting lowering of the scores.

The subject of the composition of the present invention for improving or maintaining QOL is typically a human, but is not limited to humans and may be any other non-human animal, such as a mammal other than humans. Examples of animals for which improving or maintaining QOL is required among mammals other than humans include domestic animals such as cattle, pigs, and horses and pet animals such as dogs and cats.

The subject of the composition of the present invention for improving or maintaining QOL is a subject in need of improving or maintaining QOL, and may be a subject with normal QOL (e.g., a healthy human) or a subject with enhanced QOL, or a subject with lowered QOL or a possibility of lowered QOL because of a physical or mental factor. However, it is preferable that the subject of the composition of the present invention for improving or maintaining QOL be not affected by hepatitis.

The composition of the present invention for improving or maintaining QOL may be a composition in the form of, for example, a pharmaceutical product, a food or beverage product, a feed, a food additive, or a feed additive, and is preferably in the form of a pharmaceutical product or a food or beverage product. Food or beverage products include forms of foods with functional claims, foods for specified health uses, and nutritional supplements.

The composition of the present invention for improving or maintaining QOL is preferably in the form of a composition that is ingested or administered via oral or transnasal administration, and is more preferably in the form of a composition that is ingested or administered via oral administration.

In the present invention, the term “daily intake” is used to mean the total amount of the composition of the present invention that is ingested or administered per day, and is used to preferably mean the total amount of the composition of the present invention that is ingested by or administered to one human, in particular, one adult human, per day. A specific example of the “daily intake” of the composition of the present invention when ingested or administered via oral or transnasal administration, preferably via oral administration, is 0.1 g to 500 g as the amount of the composition of the present invention. The composition of the present invention may be ingested or administered continuously, or ingested or administered when needed.

The shape of the composition of the present invention is not limited, and may be, for example, any shape such as liquid, fluid, gel, semi-solid, and solid.

The composition of the present invention may further comprise at least one additional component, in addition to the above active compound or the above turmeric extract. Preferable examples of the at least one additional component that may be comprised in the composition of the present invention include, but are not limited to, components that are acceptable in the final form, such as a pharmaceutical product, a food or beverage product, a feed, a food additive, and a feed additive, and are orally ingestible.

Examples of the additional component include sweeteners, acidulants, vitamins, minerals, thickeners, emulsifiers, antioxidants, and water. According to need, a pigment, an aroma chemical, a preservative, an antiseptic agent, a fungicide, or an additional physiologically active substance or the like may be added.

Examples of sweeteners include: monosaccharides and disaccharides, such as glucose, fructose, sucrose, lactose, maltose, palatinose, trehalose, and xylose; isomerized glucose syrup (e.g., glucose-fructose syrup, fructose-glucose syrup, and an isomerized sugar mixture), sugar alcohols (e.g., erythritol, xylitol, lactitol, Palatinit, sorbitol, and reduced starch syrup), honey, and high-intensity sweeteners (e.g., sucralose, acesulfame potassium, thaumatin, stevia, and aspartame).

Examples of acidulants include citric acid, malic acid, gluconic acid, tartaric acid, lactic acid, phosphoric acid, and salts thereof. One of these or two or more thereof can be used.

Examples of vitamins include vitamin A, vitamin B1, vitamin B2, vitamin B6, vitamin E, niacin, and inositol.

Examples of minerals include calcium, magnesium, zinc, and iron.

Examples of thickeners include carrageenan, gellan gum, xanthan gum, gum Arabic, tamarind gum, guar gum, Locust bean gum, karaya gum, agar, gelatin, pectin, soybean polysaccharides, and carboxymethyl cellulose (CMC).

Examples of emulsifiers include glycerin fatty acid ester, sucrose fatty acid ester, sorbitan fatty acid ester, lecithin, plant sterol, and saponin.

Examples of antioxidants include vitamin C, tocopherol (vitamin E), and enzyme-treated rutin.

The additional components can be adequately blended in such amounts that those skilled in the art generally employ for compositions for food or beverage products, pharmaceutical products, or others.

In the present invention, the form of the composition formulated with the above active compound or the above turmeric extract and at least one additional component by an adequate means may be the form of any of solid compositions including powders, granules, capsules, and tablets (including coated tablets such as sugar-coated tablets, multilayer tablets, orally disintegrating tablets, and chewable tablets), or the form of any of liquid compositions including solutions.

The composition of the present invention for improving or maintaining QOL preferably does not comprise one or more plants belonging to a genus selected from the genus Cordia and the genus Annona. Examples of plants belonging to the genus Cordia include, in particular, yellow cordia (Cordia lutea), specifically, flowers of yellow cordia. Examples of plants belonging to the genus Annona include, in particular, soursop (Annona muricata). Such a plant may be in the form of a part thereof or an extract thereof.

### <Composition Comprising, as Active Ingredient, at Least One of Turmeronol A, Turmeronol B, and Bisacurone, for Improving or Maintaining QOL>

The content of the above active compound or the above turmeric extract comprising it in the composition of the present invention for improving or maintaining QOL is not limited, and may be any content effective for improving or maintaining QOL.

The composition for improving or maintaining QOL according to the present invention comprises preferably 20 μg or more, more preferably 37 μg or more, particularly preferably 55 μg or more, further preferably 74 μg or more, most preferably 100 μg or more of turmeronol A and turmeronol B in total per daily intake. In this case, the composition for improving or maintaining QOL according to the present invention has particularly high effect of improving or maintaining QOL. The upper limit of the total content of turmeronol A and turmeronol B is not limited, and the composition for improving or maintaining QOL according to the present invention comprises typically 1100 μg or less, preferably 550 μg or less, more preferably 330 μg or less, particularly preferably 220 μg or less, further preferably 170 μg or less, most preferably 125 μg or less of turmeronol A and turmeronol B in total per daily intake. If at least one of turmeronol A and turmeronol B in the composition for improving or maintaining QOL according to the present invention is in the form of a turmeric extract comprising it, it is preferable for the composition to comprise the turmeric extract in such a manner that the total amount of turmeronol A and turmeronol B falls within the above range.

The composition for improving or maintaining QOL according to the present invention comprises preferably 17 μg or more, more preferably 28 μg or more, particularly preferably 43 μg or more, further preferably 57 μg or more, most preferably 77 μg or more of turmeronol A per daily intake. In this case, the composition for improving or maintaining QOL according to the present invention has particularly high effect of improving or maintaining QOL. The upper limit of the content of turmeronol A is not limited, and the composition for improving or maintaining QOL according to the present invention comprises typically 900 μg or less, preferably 430 μg or less, more preferably 260 μg or less, particularly preferably 175 μg or less, further preferably 130 μg or less, most preferably 95 μg or less of turmeronol A per daily intake. If turmeronol A in the composition for improving or maintaining QOL according to the present invention is in the form of a turmeric extract comprising it, it is preferable for the composition to comprise the turmeric extract in such a manner that the amount of turmeronol A falls within the above range.

The composition for improving or maintaining QOL according to the present invention comprises preferably 5 μg or more, more preferably 8 μg or more, particularly preferably 12 μg or more, further preferably 16 μg or more, most preferably 22 μg or more of turmeronol B per daily intake. In this case, the composition for improving or maintaining QOL according to the present invention has particularly high effect of improving or maintaining QOL. The upper limit of the content of turmeronol B is not limited, and the composition for improving or maintaining QOL according to the present invention comprises typically 250 μg or less, preferably 123 μg or less, more preferably 74 μg or less, particularly preferably 50 μg or less, further preferably 37 μg or less, most preferably 27 μg or less of turmeronol B per daily intake. If turmeronol B in the composition for improving or maintaining QOL according to the present invention is in the form of a turmeric extract comprising it, it is preferable for the composition to comprise the turmeric extract in such a manner that the amount of turmeronol B falls within the above range.

The composition for improving or maintaining QOL according to the present invention comprises preferably 80 μg or more, more preferably 133 μg or more, particularly preferably 200 μg or more, further preferably 267 μg or more, most preferably 360 μg or more of bisacurone per daily intake. In this case, the composition for improving or maintaining QOL according to the present invention has particularly high effect of improving or maintaining QOL. The upper limit of the content of bisacurone is not limited, and the composition for improving or maintaining QOL according to the present invention comprises typically 4000 μg or less, preferably 2000 μg or less, more preferably 1200 μg or less, particularly preferably 800 μg or less, further preferably 600 μg or less, most preferably 440 μg or less of bisacurone per daily intake. If bisacurone in the composition for improving or maintaining QOL according to the present invention is in the form of a turmeric extract comprising it, it is preferable for the composition to comprise the turmeric extract in such a manner that the amount of bisacurone falls within the above range.

In the composition for improving or maintaining QOL according to the present invention, the content of curcumin is typically less than 30 mg, preferably 5 mg or less, more preferably 2.5 mg or less, particularly preferably 1.5 mg or less, further preferably 1 mg or less, still further preferably 700 μg or less, and most preferably 520 μg or less per daily intake. In this case, the composition for improving or maintaining QOL according to the present invention has particularly high effect of improving or maintaining QOL. The composition for improving or maintaining QOL according to the present invention does not need to comprise curcumin, but comprises typically 94 μg or more, preferably 157 μg or more, more preferably 235 μg or more, particularly preferably 314 μg or more, most preferably 423 μg or more of curcumin per daily intake. If at least one of turmeronol A, turmeronol B, and bisacurone in the composition of the present invention for improving or maintaining QOL is in the form of a turmeric extract comprising it, it is preferable for the composition to comprise the turmeric extract in such a manner that the amount of curcumin falls within the above range.

The composition for improving or maintaining QOL according to the present invention is preferably a composition that is continuously ingested, specifically, a composition that is continuously ingested at a frequency of once or twice or more per day, preferably over 4 weeks or more, more preferably over 8 weeks or more, most preferably over 12 weeks or more.

The composition for improving or maintaining QOL according to the present invention may be the above active compound or the above turmeric extract comprising it, per se, or a composition comprising the above active compound or the above turmeric extract comprising it and at least one additional component. When the composition of the present invention comprises the above active compound or the above turmeric extract comprising it and at least one additional component, the composition may be a composition obtained by mixing the above active compound or the above turmeric extract comprising it and at least one additional component, or a composition obtained by formulating with the active compound or the turmeric extract comprising it and at least one additional component by an adequate means, or a composition obtained by mixing a composition obtained by formulating with the active compound or the turmeric extract comprising it and at least one additional component with another additional component.

### <Method for Improving or Maintaining QOL by Administration of at Least One of Turmeronol A, Turmeronol B, and Bisacurone>

A further aspect of the present invention relates to a method for improving or maintaining QOL, comprising:

administering at least one of turmeronol A, turmeronol B, and bisacurone, or a turmeric extract comprising at least one of turmeronol A, turmeronol B, and bisacurone and extracted with at least one extraction solvent selected from the group consisting of water and a hydrophilic organic solvent, to a subject in need of improving or maintaining QOL; and improving or maintaining QOL in the subject.

The active compound or the turmeric extract for use in the method according to the present aspect can be in the aforementioned form of the composition of the present invention. The method according to the present aspect may be a medical method or a nonmedical method.

The subject in the method according to the present aspect is typically a human, but may be a mammal other than humans. The subject may be a domestic animal or a pet animal as described above. The subject in need of improving or maintaining QOL may be a subject with normal QOL (e.g., a healthy human) or a subject with enhanced QOL, or a subject with lowered QOL or a possibility of lowered QOL because of a physical or mental factor.

In the method according to the present aspect, the administration route is preferably oral or transnasal administration, and is particularly preferably oral administration.

In the method according to the present aspect, the amount of the active compound or the turmeric extract administered to the subject is not limited, and may be any amount effective for improving or maintaining QOL. For example, in one embodiment, in the case that the subject is an adult human, it is preferable to administer the active compound or the turmeric extract to the subject, so that 20 μg or more of turmeronol A and turmeronol B in total can be administered to the subject per day. In another embodiment, in the case that the subject is an adult human, it is preferable to administer the active compound or the turmeric extract to the subject, so that 17 μg or more of turmeronol A can be administered to the subject per day. In another embodiment, in the case that the subject is an adult human, it is preferable to administer the active compound or the turmeric extract to the subject, so that 5 μg or more of turmeronol B can be administered to the subject per day. In another embodiment, in the case that the subject is an adult human, it is preferable to administer the active compound or the turmeric extract to the subject, so that 80 μg or more of bisacurone can be administered to the subject per day. Further, it is preferable to administer the active compound or the turmeric extract to the subject, so that the daily dose of curcumin can be less than 30 mg. Further preferably, it is preferable to administer the active compound or the turmeric extract to the subject, so that each component of the active compound can be administered to the subject per day at a weight that is described as a preferred amount for the component of the active compound in the daily intake of the composition for improving or maintaining QOL according to the present invention.

### EXAMPLES

### 1. Method for Production of Turmeric Extract

A turmeric extract was prepared by extracting the rhizome part of turmeric (Curcuma longa) with water, and heat-drying the resulting extract under reduced pressure to remove water. The amounts of turmeronol A (TA) and turmeronol B (TB) in the turmeric extract were measured by using LC/MS, and the amounts of bisacurone and curcumin were measured by using HPLC.

### 2. Test Food

The test food was a tablet comprising the turmeric extract containing, per three tablets, 86.5 μg of TA, 24.7 μg of TB, 400 μg of bisacurone, and 471 μg of curcumin. The tablet was prepared by mixing the turmeric extract, an excipient, a lubricant, a coloring agent, and other ingredients for manufacturing (fine grain silicon dioxide and sucrose fatty acid ester), and tableting the mixture.

The placebo food was a tablet prepared with replacing the turmeric extract in the test food with an excipient.

### 3. Evaluation Method for QOL

MOS 36-Item Short-Form Health Survey (SF-36 (Registered Trademark)), a validated survey method for evaluating health-related QOL in humans, is widely used to assess health condition for a wide variety of individuals including healthy and sick individuals (Shunichi FUKUHARA, Yoshimi SUZUKAMO (2015) “SF-36v2 manual, Japanese version”, iHOPE International).

In the present experiment, test subjects were allowed to answer questions in a questionnaire system according to SF-36v2 (Registered Trademark) (acute version), and QOL was evaluated with a method of scoring general health (GH), vitality (VT), mental health (MET), and mental component summary (MCS) on the basis of the results.

### 4. Evaluation Test for QOL

Eighty-seven 50- to 69-year-old male and female test subjects were randomly assigned to two groups (placebo group: 44 test subjects, turmeric extract group: 43 test subjects), and, under a double-blind test, allowed to ingest three tablets of the test food, which contained the turmeric extract, or the placebo food, which did not contain the extract, once a day before supper for 12 weeks. The test subjects were allowed to answer questions in a questionnaire system according to SF-36v2 (Registered Trademark) (acute version) before the intake of the test food, and 4 weeks, 8 weeks, and 12 weeks after the intake, and general health (GH), vitality (VT), mental health (MH), and mental component summary (MCS) were scored on the basis of the results.

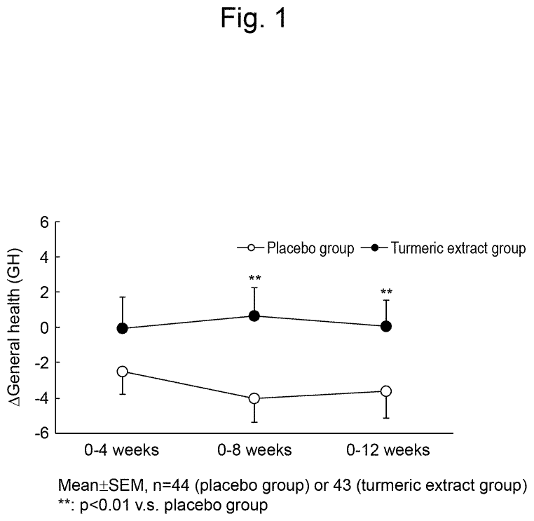

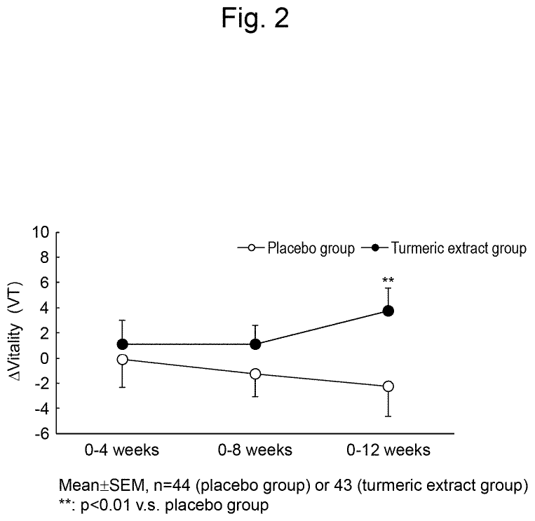

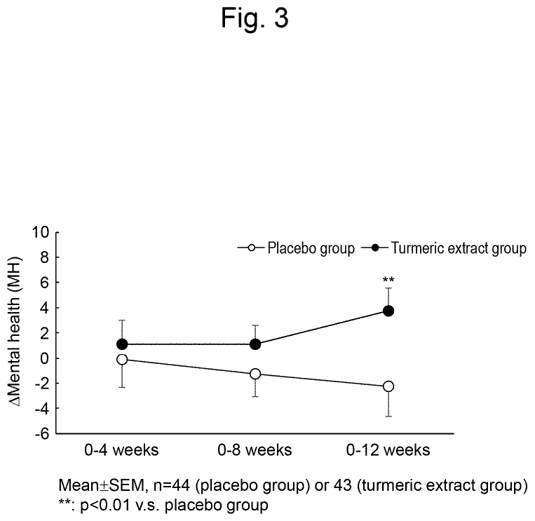

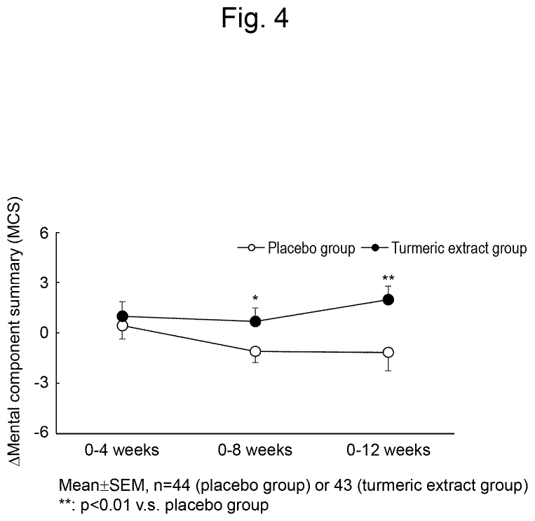

FIGS. 1, 2, 3, and 4 respectively show amounts of change (ΔGH, ΔVT, ΔMH, ΔMCS) in general health (GH), vitality (VT), mental health (MH), and mental component summary (MCS) scores from before the intake of the test food (the turmeric extract group or the placebo group) to 4 weeks, 8 weeks, and 12 weeks after the intake. As demonstrated in the figures, the amounts of change in general health (GH) and the amounts of change in mental component summary (MCS) from before the initiation of intake of the test food to 8 weeks and 12 weeks thereafter in the turmeric extract group were both significantly more than those in the placebo group. In addition, the vitality (VT) and the mental health (MH) on week 12 in the turmeric extract group were both significantly higher than those in the placebo group. These results revealed that intake of the turmeric extract improves QOL.

All the publications, patents, and patent applications cited in the present description are incorporated herein by reference in their entirety.

1-10. (canceled)

11. A method for improving or maintaining quality of life (QOL), comprising:
administering at least one of turmeronol A, turmeronol B, and bisacurone, to a subject in need of improving or maintaining quality of life (QOL); and
improving or maintaining quality of life (QOL) in the subject.

12. The method according to claim 11, wherein the administering comprises administering a turmeric extract comprising the at least one of turmeronol A, turmeronol B, and bisacurone and extracted with at least one extraction solvent selected from the group consisting of water and a hydrophilic organic solvent, to the subject.

13. The method according to claim 11, wherein the administering comprises administering the least one of turmeronol A, turmeronol B, and bisacurone, without being in combination with one or more plants belonging to a genus selected from the genus Cordia and the genus Annona, to the subject.

14. The method according to claim 11, wherein the subject is not affected by hepatitis.

15. The method according to claim 14, wherein the subject is a healthy human.

16. The method according to claim 11, wherein the administering comprises administering 20 μg or more of turmeronol A and turmeronol B in total to the subject per day.

17. The method according to claim 11, wherein the administering comprises administering 17 μg or more of turmeronol A to the subject per day.

18. The method according to any claim 11, wherein the administering comprises administering 5 μg or more of turmeronol B to the subject per day.

19. The method according to claim 11, wherein the administering comprises administering 80 μg or more of bisacurone to the subject per day.

20. The method according to claim 11, wherein the administering comprises administering the at least one of turmeronol A, turmeronol B, and bisacurone, so that the daily dose of curcumin is less than 30 mg.
